(19) United States
(12) Patent Application Publication
(10) Pub. No.: US 2008/0097330 A1
(43) Pub. Date: Apr. 24, 2008

(54) CATHETER INSERTION DEVICE WITH FLUID LEAKAGE CONTROL

(75) Inventors: Alan D. King, Burlington, CT (US); Thomas K. Sutton, West Simsbury, CT (US)

Correspondence Address: WOOD, HERRON & EVANS, LLP, 2700 CAREW TOWER, 441 VINE STREET, CINCINNATI, OH, 45202, US

(73) Assignee: SMITHS MEDICAL ASD, INC., Keene, NH (US)

(21) Appl. No.: 11/458,297

(22) Filed: Jul. 18, 2006

(51) Int. Cl. A61M 25/06

(52) U.S. Cl. 604/164.02

(57) ABSTRACT

A catheter insertion device is provided in which a needle cannula thereof has one or more markers to provide a visual and/or tactile indication to a user that the needle cannula has been withdrawn from a position in which the sharp tip thereof is exposed beyond the catheter tube to a position wherein a part of the cannula needle shaft and the tip are within a proximal portion of the catheter tube so as to impede fluid leakage through the tube, but removed from a distal portion of the tube to free it to flex.

### FIELD OF THE INVENTION

The present invention relates to catheter insertion devices, and more particularly, to controlling fluid leakage during use thereof to insert the catheter.

### DESCRIPTION OF PRIOR ART

A catheter typically has a catheter hub with a flexible catheter tube extending therefrom, the distal end of which is to be placed into a bodily space, such as within a blood vessel or an epidural space of a patient. A rigid needle cannula is provided having a needle shaft terminating in a beveled portion defining a sharp tip. The shaft is received through the hub and tube of the catheter such that the sharp tip is exposed at the distal end of the catheter tube. The sharp tip pierces the skin and tissue of the patient, and enters the bodily space in which the tube end is to be positioned. The shaft provides rigidity to carry the tube to the distal end through the skin and tissue and into the bodily space.

Once the tube is inserted into the bodily space, medical practitioners often desire to partially withdraw the rigid needle cannula so that the distal portion of the tube is free to flex, while the shaft is still within a proximal portion of the tube to provide resistance to fluid leakage through the catheter. The medical practitioner may then take advantage of the flexibility of the distal portion of the tube for final positioning thereof in the bodily space. If the medical practitioner withdraws the needle shaft until the beveled portion starts to or does pass beyond the tube (i.e., into the hub or proximally beyond the hub), however, there is an open fluid leakage path, such as for blood or other bodily fluid, during the final placement of the catheter tube.

Typically, the medical practitioner tries to avoid such removal of the shaft from the catheter tube by visually monitoring the catheter as the needle cannula is withdrawn. Visual monitoring is imprecise and unreliable, and may in some cases be impractical depending upon how the catheter is constructed or how the device is held by the user. The needle shaft may thus end up being withdrawn from the tube before the medical practitioner realizes the tube has become unblocked. In such cases, it may be necessary to re-insert the needle shaft back into the proximal portion of the catheter tube. Such re-insertion is not always possible or practical, and in any event comes after a risk of fluid leakage has already occurred.

The problems can be compounded with safety catheter insertion devices. Many safety catheter insertion devices include a safety device with a portion that is resiliently urged or spring-biased against the needle shaft and which closes down over or grips the needle shaft to reduce the risk of exposure to the sharp needle tip after use. One example is shown in Bialecki et al U.S. Pat. No. 6,652,486 in which the safety device is a clip within the catheter hub which closes down over the sharp tip of the catheter of the needle cannula when it is withdrawn from the catheter tube and into the catheter hub. In some safety catheter insertion devices, a housing extends from the catheter hub and contains the safety device. Examples are shown in co-pending U.S. patent application Ser. No. 11/161,554, filed Aug. 8, 2005 and Sircom U.S. Pat. No. 5,322,517, in which a canting plate grips the needle shaft when the needle tip passes sufficiently into the housing. Such safety devices might reduce or fully occlude visualization of the needle shaft, or might interfere with re-insertion should the needle cannula be withdrawn beyond the catheter tube.

### SUMMARY OF THE INVENTION

The present invention provides the medical practitioner with the ability to reliably withdraw the needle cannula sufficiently to free the distal portion of the tube to flex so as to facilitate final placement of the tube in the bodily space while maintaining at least a portion of the shaft proximal of the sharp tip within a proximal portion of the tube to continue to impede fluid leakage. To that end, and in accordance with the principles of the present invention, a marking is associated with an area of the needle cannula shaft proximal of the tip an amount sufficient (a) to place the marker within the catheter, and advantageously within the catheter tube, when the sharp tip of the needle cannula is exposed for catheter insertion, but (b) to place the marker exposed beyond the tube, at least in the catheter hub and possibly exposed beyond the catheter hub, when the shaft has been sufficiently withdrawn so as to be removed from the distal portion of the tube, but still within the proximal portion thereof such that there is still a resistance to the fluid leakage.

The marker may be a visual marker. The area of the shaft with which the visual marker is associated is advantageously one that causes the marker to be exposed beyond at least the catheter hub, and further to be exposed beyond the housing if there is one, so as to provide visual feedback to the medical practitioner to discontinue the proximal withdrawal of the needle cannula before a free flow fluid leakage path is established. The visual marker thus informs the user that the shaft has been sufficiently withdrawn to free the distal tube end to flex, while a portion of the shaft is still in the proximal tube portion to impede fluid leakage. The visual marker can be a material on the shaft such as ink applied to the area, or could be a surface affect of the shaft such as by a grind, filing, etch, or knurl in the area.

Alternatively, or additionally, the marker could be a tactile marker which cooperates with the resiliently-urged or spring-biased portion of the safety device to provide tactile feedback to the medical practitioner when the needle cannula has been sufficiently withdrawn. The tactile marker is a disruption of the surface of the needle shaft in the area sufficient to cause a sensation to be felt by the user as the marker passes along the portion of the safety device that normally bears against the shaft. The disruption could be the result of a crimp or coining of the shaft in the area (which may produce an outward projection), or could be the result of grinding, filing, etching, machining or milling (which may produce a surface roughening and/or in an inward recess or the like). The location of the area for the tactile marker is one that brings the marker into the hub in those products where the resiliently-urged or spring-biased portion of the safety device is in the hub. For those products where the resiliently-urged or spring-biased portion of the safety device is in the housing, the location of the area for the tactile marker is one that brings the marker into the housing.

The marker could be both visual and tactile, or there could be two areas, each with a marker, one being primarily visual and the other being primarily tactile. Where both a visual and a tactile marker are to be used, the tactile marker is advantageously in an area of the shaft between the needle tip and the area of the visual marker so that both markers are positioned for their respective purposes. With some safety catheter insertion devices, such as shown in aforementioned Bialecki et al U.S. Pat. No. 6,652,486, there is already an outwardly projecting retaining disruption of the needle shaft which is sized to prevent proximal withdrawal of the needle tip from the safety device. While such a disruption would be able to provide a tactile sensation to the user, it is understood that the dimensional requirements involved cause the sensation to occur after the shaft has been withdrawn from the tube. In the present invention, the tactile marker would be in an area proximal of the retaining disruption and would be sized so as not to prevent proximal movement of the needle cannula or to release and/or activate the safety device.

In many catheter insertion devices, the needle cannula is hollow and thus has a fluid lumen therethrough. The marker(s) of the present invention advantageously will not extend through the needle shaft surface so as to open a fluid path into the fluid lumen thereat. Otherwise, as the marker becomes exposed beyond the tube, an alternate leakage path would be created.

By virtue of the foregoing, there is thus provided a catheter insertion device which provides the medical practitioner with the ability to reliably withdraw the needle cannula sufficiently to free the distal portion of the tube to flex so as to facilitate final placement of the tube in the bodily space while maintaining at least a portion of the shaft proximal of the sharp tip within a proximal portion of the tube to continue to impede fluid leakage. These and other objects and advantages of the present invention shall be made apparent from the accompanying drawings and the description thereof.

### BRIEF DESCRIPTION OF THE DRAWINGS

The accompanying drawings, which are incorporated in and constitute a part of this specification, illustrate embodiments of the invention, and together with the general description of the invention given above, and the detailed description of the embodiments given below, serve to explain the principles of the present invention.

FIG. 1 is a cross-sectional view of a catheter insertion device in accordance with the principles of the present invention, showing the needle cannula in a first position with a sharp tip exposed ready for insertion of the device;

FIG. 2 is a view of the catheter insertion device of FIG. 1 showing the needle cannula in a second position removed from the catheter tube;

FIG. 3 is a cross-sectional view of the catheter insertion device of FIG. 1 showing the needle cannula in a third position with the sharp tip and a portion of the shaft proximal of the sharp tip still within the catheter tube for purposes of explaining the principles of the present invention;

FIG. 4 is a cross-sectional view of an embodiment of a safety catheter insertion device in accordance with the principles of the present invention, showing the needle cannula in the first position with the sharp tip exposed ready for insertion of the device;

FIG. 5 is a cross-sectional view of the safety catheter insertion device of FIG. 4 showing the needle cannula in the second position from the catheter tube;

FIG. 6 is a cross-sectional view of the safety catheter insertion device of FIG. 4 showing the needle cannula in the third position with the sharp tip and a portion of the shaft proximal of the sharp tip still within the catheter tube for purposes of explaining the principles of the present invention;

FIG. 7 is a cross-sectional view of another embodiment of a safety catheter insertion device in accordance with the principles of the present invention, showing the needle cannula in the first position with the sharp tip exposed ready for insertion of the device;

FIG. 8 is a cross-sectional view of the safety catheter insertion device of FIG. 7 showing the needle cannula in the second position removed from the catheter tube; and

FIG. 9 is a cross-sectional view of the catheter insertion device of FIG. 7 showing the needle cannula in the third position with the sharp tip and a portion of the shaft proximal of the sharp tip still within the catheter tube for purposes of explaining the principles of the present invention.

### DETAILED DESCRIPTION OF THE DRAWINGS

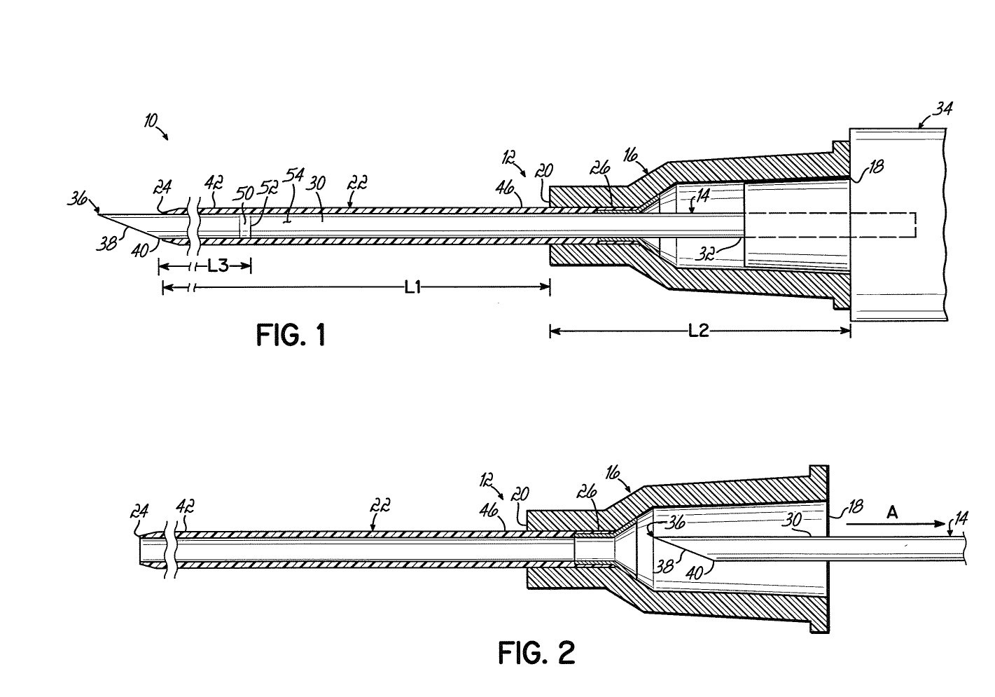

With reference to FIGS. 1-3, there is shown a catheter insertion device 10 including a catheter 12 and a needle cannula 14. Catheter 12 includes a catheter hub 16 having a proximal opening 18 advantageously conforming to luer standards and a distal end 20 from which distally extends a flexible catheter tube 22 terminating in open, beveled distal end 24. Tube 22 may extend proximally into catheter hub 16 and is secured thereto, such as with an eyelet 26. Tube 22 extends a distance L1 from hub distal end 20 to tube distal end 24. Hub 16 has a length L2 between proximal opening 18 and distal end 20.

Needle cannula 14 includes a rigid shaft 30 with its proximal end 32 supported by needle hub 34 which may be or couple to a flash chamber. The needle shaft 30 terminates in a distal sharp tip 36 defined by a bevel 38 which starts at heel 40.

As shown in FIG. 1, needle cannula 14 has a first position in which the shaft 30 extends through hub 16 and tube 22 with sharp tip 36 extending distally of opening 24 of tube 22 to facilitate insertion of device 10 into a patient (not shown) so as to access a bodily space such as a blood vessel, epidural space, or the like (also not shown). Needle hub 34 may also have a portion that advantageously conforms to luer standards and engages with or within opening 18 of hub 16 in the first position of needle cannula 20. In the first position of needle cannula 14, catheter insertion device 10 may be used to insert catheter tube 22 into a bodily space (not shown), as will be readily understood.

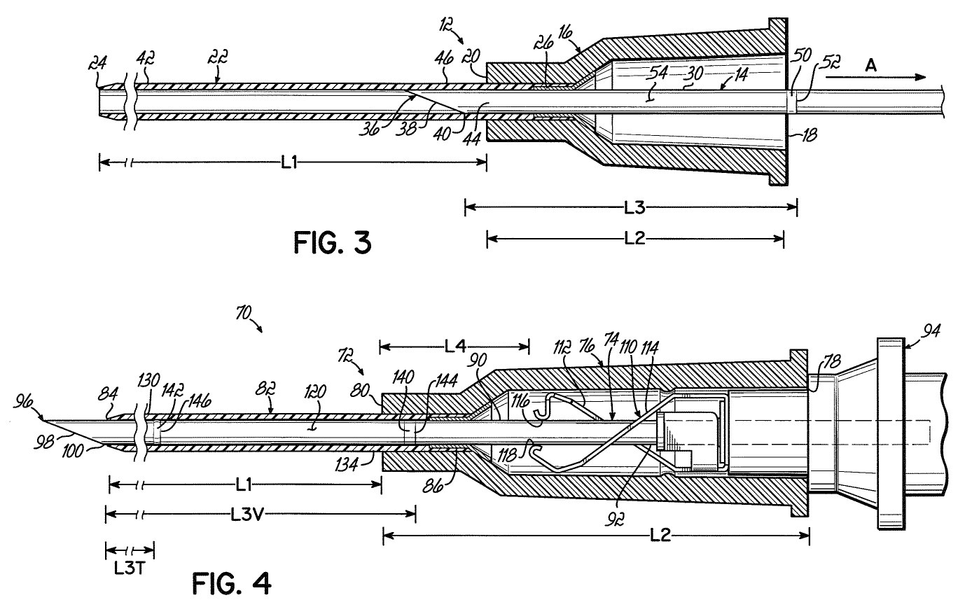

After insertion of catheter tube 22, the medical practitioner (not shown) will withdraw needle cannula 14 in the direction of arrow A (FIGS. 2 and 3). Ultimately, needle cannula 14 is to be withdrawn, at least beyond catheter tube 22, so as to define a second position of needle cannula 14 as seen in FIG. 2. To that end, the shaft 30 and the sharp tip end 36 are completely removed from at least tube 22, and may further be removed from catheter hub 16. After removal, hub 16 may be coupled to a source of fluid or to a medical device (both not shown) via opening 18 as is conventional.

In accordance with the principles of the present invention, it is desired that the medical practitioner be able to stop withdrawal of needle cannula 14 before reaching the second position, so as to stop in a third position of needle cannula 14 in which needle shaft 30 has been withdrawn from a distal portion 42 of catheter tube 22 while a portion 44 of shaft 30 proximal of heel 40 remains within a proximal portion 46 of tube 22. In this third position of needle cannula 14, the distal portion 42 is now free to flex for final placement of tube end 24 within the bodily space (not shown), yet there is still an impediment to fluid leakage through tube 22 into hub 16. To allow the medical practitioner to reliably move needle cannula 14 from the first position to the third position without going past the third position into the second position, a marker 50 is associated with shaft 30 in an area 52 thereof sufficiently proximal of heel 40 so as to position marker 50 within catheter 12, and advantageously within tube 22 as shown in FIG. 1, in the first position of needle cannula 14, but exposed beyond hub 16, such as just proximally of opening 18, in the third position of needle cannula 14 as seen in FIG. 3. To that end, area 52 is proximally spaced from heel 40 a distance L3, which is advantageously greater than L2 and less than the sum of L1 plus L2. For many catheters, L3 is more advantageously, less than L1. For short catheters, however, L3 may be more advantageously greater than L1. The foregoing advantageously places marker 50 within catheter 12 and possibly within catheter tube 22 depending on the length thereof, in the first position of needle cannula 14, and exposed beyond hub 16 in the third position of needle cannula 14.

In the catheter insertion device 10 of in FIGS. 1-3, marker 50 is advantageously a visual marker such that the medical practitioner may visualize emergence of marker 50 as it passes beyond (i.e., is exposed beyond) the opening 18 of hub 16 whereat the medical practitioner should stop withdrawal of needle cannula 14 along the direction of arrow A to thus reliably position needle cannula 14 in the desired third position. At that position, the medical practitioner may then finalize placement of catheter tube 22, and particularly distal portion 42 and/or opening 24 thereof, within the bodily space, followed, advantageously, by withdrawal of needle cannula 14 to at least the second position shown in FIG. 2.

The visual marker 50 may be associated with shaft 30 by applying a visually perceptible material such as ink to the surface 54 of shaft 30 at or up to area 52, or by a visually perceptible surface affect at or up to area 52 such as grinding, filing, etching, knurling, or otherwise altering the visually perceptible appearance of surface 54.

In use, catheter 12 is inserted into a bodily space (not shown) with needle cannula 14 in the first position. Once inserted, needle cannula 14 is withdrawn until at least some portion of marker 50 is seen proximal of hub 16, at which time needle cannula 14 is in the third position. Withdrawal of needle cannula 14 is stopped, allowing catheter distal portion 42 and/or tube end 24 to be finally positioned in the bodily space. Thereafter, needle cannula 14 may be withdrawn to the second position and removed from catheter 12 for subsequent medical use of catheter 12.

With reference to FIGS. 4-6, there is shown one embodiment of a safety catheter insertion device 70 including a catheter 72 and a needle cannula 74. Catheter 72 includes a catheter hub 76 having a proximal opening 78 advantageously conforming to luer standards and a distal end 80 from which distally extends a flexible catheter tube 82 terminating in open, beveled distal end 84. Tube 82 may extend proximally into hub 76 and is secured thereto, such as with an eyelet 86.

Needle cannula 74 includes a rigid shaft 90 with its proximal end 92 supported by needle hub 94 which may be or couple to a flash chamber. The needle shaft 90 terminates in a distal sharp tip 96 defined by a bevel 98 which starts at heel 100.

Situated within hub 76, distal of opening 78 and proximal of eyelet 86, is a safety device 110 in the form of a clip. The structure and operation of clip 110, and its cooperation with hub 76 and needle cannula 74, will not be detailed here as they are explained in Bialecki U.S. Pat. No. 6,652,486, the disclosure of which is incorporated herein by reference in its entirety.

As shown in FIG. 4, needle cannula 74 has a first position in which the shaft 90 extends through hub 76 and clip 110 therein, and through tube 82 with sharp tip 96 extending distally of opening 84 of tube 82 to facilitate insertion of device 70 into a patient (not shown) so as to access a bodily space such as a blood vessel, epidural space, or the like (also not shown). Needle hub 94 may also have a portion that advantageously conforms to luer standards and engages with or within opening 78 of hub 76 in the first position of needle cannula 74. Clip 110 has a pair of arms 112, 114, each with a portion 116, 118 respectively, between which shaft 90 passes. In the first position of needle cannula 74, portions 116, 118 are resiliently-urged against opposed aspects of surface 120 of shaft 90. In that first position, catheter insertion device 70 may be used to insert catheter tube 82 into a bodily space, as will be readily understood.

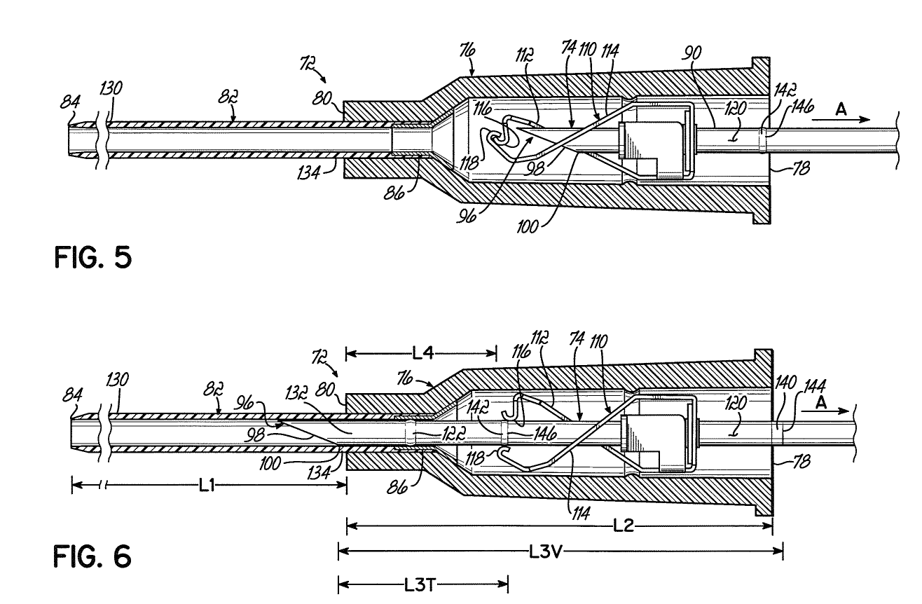

After insertion of catheter tube 82, the medical practitioner (not shown) will withdraw needle cannula 74 in the direction of arrow A (FIGS. 5 and 6). Ultimately, needle cannula 74 is to be withdrawn, at least beyond catheter tube 82, so as to pass needle tip 96 beyond portions 116, 118 to define a second position of needle cannula 74 as seen in FIG. 5. As or after needle tip 76 passes beyond portions 116, 118, arms 112, 114 can cause tip 96 to be enclosed by clip 110. To that end, shaft 90 may include an outwardly projecting disruption 122 (FIG. 6) of surface 120 positioned and sized to prevent proximal removal of needle cannula 74 from clip 110 once tip 96 is enclosed thereby and to cooperate in removal of clip 110 from catheter hub 76. Shaft 90 and the sharp tip end 96 can be completely removed from at least tube 92, and, with tip 96 enclosed by clip 110, may further be removed from catheter hub 76, so as to be removed completely from catheter 72 (along with clip 110). After removal of needle cannula 74 from catheter 72, hub 76 may be coupled to a source of fluid or to a medical device (both not shown) via opening 78 as is conventional.

In accordance with the principles of the present invention, it is desired that the medical practitioner be able to stop withdrawal of needle cannula 74 before reaching the second position, so as to stop in a third position of needle cannula 74 in which needle shaft 90 has been withdrawn from a distal portion 130 of catheter tube 82 while a portion 132 of shaft 90 proximal of heel 100 remains within a proximal portion 134 of tube 82. In this third position of needle cannula 74, portions 116, 118 are still resiliently urged against surface 120 of shaft 90. Also in this third position of needle cannula 74, the distal portion 130 of tube 82 is now free to flex for final placement of tube end 84 within the bodily space (not shown), yet there is still an impediment to fluid leakage through tube 82 into hub 76. To allow the medical practitioner to reliably move needle cannula 74 from the first position to the third position without going past the third position into the second position, one or both of a first marker 140 and a second marker 142 are associated with shaft 90 in respective areas 144 and 146 thereof which are each sufficiently proximal of heel 100 so as to be within catheter 72, and advantageously within tube 82 as shown in FIG. 4, in the first position of needle cannula 74, but so that, in the third position of needle cannula 74, first marker 140 is exposed beyond hub 76 such as just proximally of opening 78 and/or second marker 142 is exposed beyond tube 82 but within hub 76 in juxtaposition to pass along one or both of resiliently urged portions 116, 118 of clip 110, all as seen in FIG. 3.

Tube 82 of catheter 72 extends a distance L1 from hub distal end 80 to tube distal end 84. Hub 76 has a length L2 between proximal opening 78 and distal end 80, with length L2 being long enough to normally enclose clip 110 in the first position of needle cannula 74. Clip 110 is typically held within hub 76 such that at least one of portions 116, 118 is spaced proximally of the distal end 80 of hub 76 by a distance L4. Areas 144 and 146 are proximally spaced from heel 100 a respective distance L3V and L3T which, in accordance with the principles of the present invention, are in respective relationships to L1, L2 and/or L4. To that end, L3V is advantageously greater than L2 and less than the sum of L1 plus L2. For many catheters 72, L3V is more advantageously less than L1, but for short catheters, may be more advantageously greater than L1. Similarly, L3T is advantageously greater than L4 and less than the sum of L1 plus L4, and more advantageously less than L1. The foregoing advantageously places at least marker 142 within catheter tube 82, and places marker 140 within catheter 72 and possibly within tube 82, in the first position of needle cannula 74, and such that they are exposed beyond at least catheter tube 82, with marker 140 further being exposed beyond hub 76, in the third position of needle cannula 74.

In the catheter insertion device 70 of in FIGS. 4-6, marker 140 is advantageously a visual marker and marker 142 is advantageously a tactile marker such that the medical practitioner may visualize emergence of marker 140 as it passes beyond (i.e., is exposed beyond) the opening 78 of hub 76 and/or may feel a tactile sensation as marker 142 passes along portion 116 or portion 118, whereat the medical practitioner should stop withdrawal of needle cannula 74 along the direction of arrow A to thus reliably position needle cannula 74 in the desired third position. At that position, the medical practitioner may then finalize placement of catheter tube 82, and particularly distal portion 130 and/or opening 84 thereof, within the bodily space, followed, advantageously, by withdrawal of needle cannula 74 to at least the second position shown in FIG. 5.

The visual marker 140 may be associated with area 144 by applying a visually perceptible material such as ink to the surface 120 of shaft 90 at or up to area 144 or by a visually perceptible surface affect at or up to area 144 by grinding, filing, etching, knurling, or otherwise altering the visually perceptible appearance of surface 120. Advantageously, marker 140 is not such as would create a noticeable tactile sensation as it passes along portions 116 and 118, and further advantageously does not create an outward projection large enough to prevent proximal withdrawal of needle cannula 74 through clip 110 to the second position or to cause release of clip 110. Tactile marker 142 may be a disruption of surface 120 in area 146 such as an outwardly directed (relative to surface 120) projection, an inwardly directed (relative to surface 120) recess or the like (such as a notch or gap, by way of example), or a roughening thereof, sufficient to interact with portion 116 and/or 118 to create a tactile feedback to the user (not shown) as marker 142 passes along the resiliently-urged portion(s). Advantageously, the disruption is an outwardly directed projection and may be associated with area 146 by crimping or coining surface 120 thereat, by way of example, although the extent of outward projection is advantageously not as large as that for disruption 122, so as not to prevent proximal withdrawal of needle cannula 74 through clip 110 into the second position or to cause release of clip 110. Where marker 142 is a roughening or an inwardly directed recess or the like (both not shown), it may be associated with area 146 by grinding, filing, etching, machining, or milling, by way of example, although marker 142 is advantageously not so far into surface 120 as to allow clip 110 to activate as marker 142 passes along portions 116 and 118. As may be seen in FIG. 6, marker 140 (and marker 142) are proximal of retaining disruption 122.

Use of safety catheter insertion device 70 is similar to catheter insertion device 10, except that withdrawal of the needle cannula 74 may be stopped at the third position by either seeing some portion of visual marker 140 exposed beyond hub 76 and/or feeling a tactile sensation by tactile marker 142 exposed beyond tube 82 within hub 76 to pass along portion 116 and/or 118.

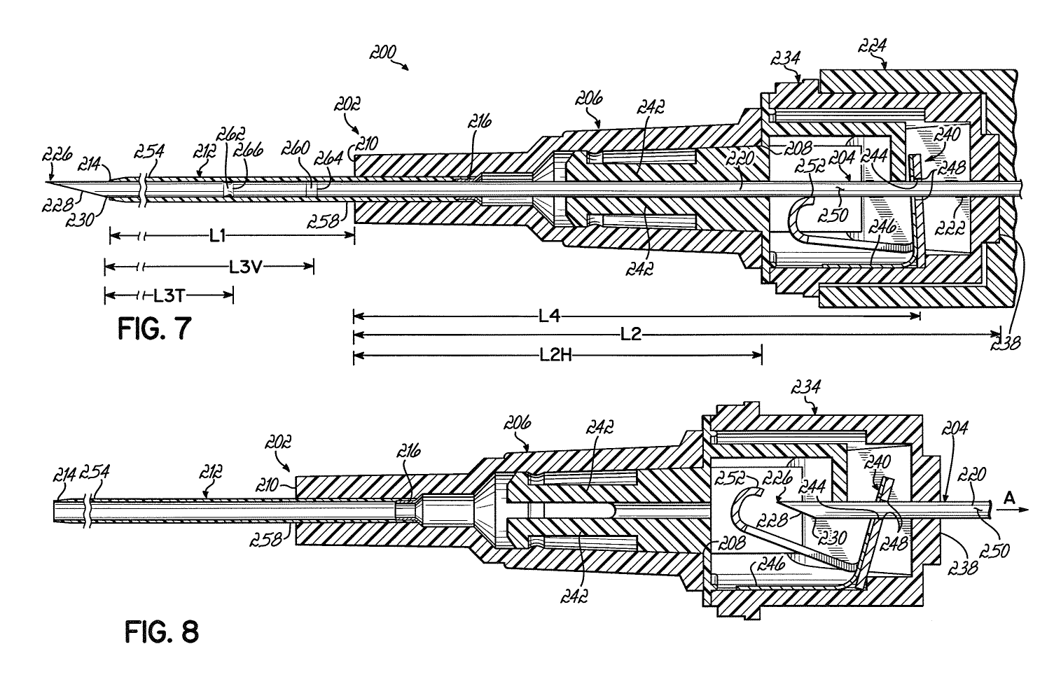

With reference to FIGS. 7-9, there is shown another embodiment of a safety catheter insertion device 200 including a catheter 202 and a needle cannula 204. Catheter 202 includes a catheter hub 206 having a proximal opening 208 advantageously conforming to luer standards and a distal end 210 from which extends a flexible catheter tube 212 terminating in open, beveled distal end 214. Tube 212 may extend proximally into hub 206 and is secured thereto, such as with an eyelet 216.

Needle cannula 204 includes a rigid shaft 220 with its proximal end 222 supported by needle hub 224 which may be or couple to a flash chamber. The needle shaft 220 terminates in a distal sharp tip 226 defined by a bevel 228 which starts at heel 230. Extending proximally from catheter hub 206, and located between hubs 206 and 224, is a housing 234. Housing 234 may be coupled distally to catheter hub 206 by a duckbill mechanism as described in co-pending U.S. patent application Ser. No. 11/161,554, filed Aug. 8, 2005, the disclosure of which is incorporated herein by reference in its entirety. Housing 234 has a proximal end 238 and includes a safety device 240 in the form of a canting plate therein. The structure and operation of housing 234 and canting plate 240, and their cooperation with catheter hub 206 and needle cannula 204, will not be detailed here as they are explained in the aforementioned incorporated-by-reference co-pending U.S. patent application Ser. No. 11/161,554.

As shown in FIG. 7, needle cannula 204 has a first position in which the shaft 220 extends through housing 234 (and duckbill arms 242 thereof) and canting plate 240 therein, through catheter hub 206, and through tube 212 with sharp tip 226 extending distally of opening 214 of tube 212 to facilitate insertion of device 200 into a patient (not shown) so as to access a bodily space such as a blood vessel, epidural space, or the like (also not shown). Housing 234 has duckbill arms 242, which extend into and engage within catheter hub 206 in the first position of needle cannula 204. Plate 240 has a through-hole 244 through which shaft 220 passes. In the first position of needle cannula 204, plate 240 is resiliently urged under the bias of spring member 246 against needle shaft 220 such that one or more of edge(s) 248 of through-hole 244 lightly bear against surface 250 of shaft 220. Also, a cantilevered edge 252 is resiliently urged against shaft 250 distal of plate 240. In that first position, catheter insertion device 200 may be used to insert catheter tube 212 into a bodily space, as will be readily understood.

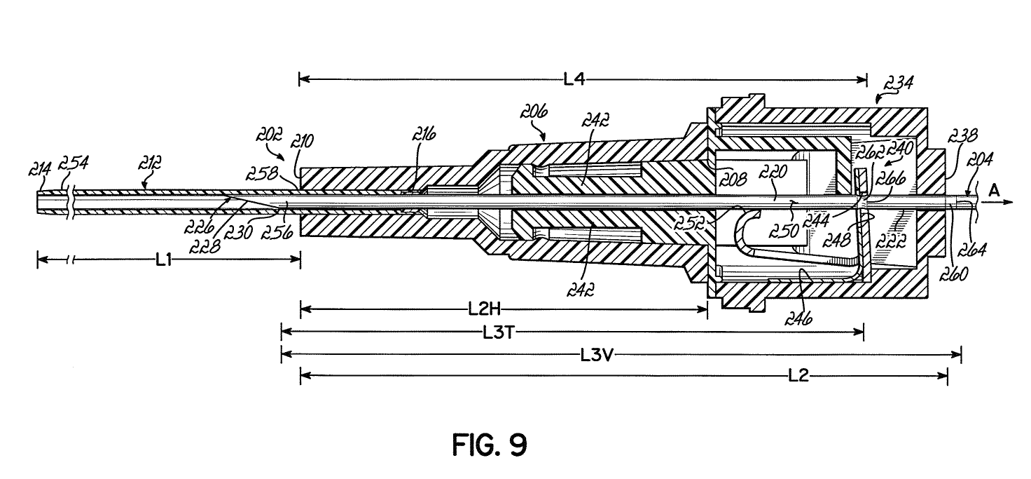

After insertion of catheter tube 212, the medical practitioner (not shown) will withdraw needle cannula 204 in the direction of arrow A (FIGS. 8 and 9). Ultimately, needle cannula 204 is to be withdrawn, at least beyond catheter tube 212, so as to pass needle tip 226 into housing 234, to define a second position of needle cannula 204, an example of which is seen in FIG. 8. Advantageously, needle tip 226 is withdrawn until it passes beyond edge 252 to allow resiliently-urged or spring-biased plate 240 to cant such that one or more of edges 248 of through-hole 244 strongly bite against the surface 250 to grip shaft 220 and secure tip 226 within housing 234. Thus, plate 240 continues to be resiliently urged against shaft 220 in the second position of needle cannula 204, whereas edge 252 is no longer urged against shaft 220. Shaft 220 and the sharp tip end 226 can be completely removed from hub 206 with tip 226 enclosed by housing 234 and held therein by plate 240, so as to be removed completely from catheter 202. After removal of needle cannula 204 from catheter 202, hub 206 may be coupled to a source of fluid or to a medical device (both not shown) via opening 208 as is conventional.

In accordance with the principles of the present invention, it is desired that the medical practitioner be able to stop withdrawal of needle cannula 204 before reaching the second position, so as to stop in a third position of needle cannula 204 in which needle shaft 220 has been withdrawn from a distal portion 254 of catheter tube 212 while a portion 256 of shaft 220 proximal of heel 230 remains within a proximal portion 258 of tube 212. In this third position of needle cannula 204, plate 240 is still resiliently urged to lightly bear against shaft 220. Also, in this third position of needle cannula 204, the distal portion 254 of the tube 212 is now free to flex for final placement of tube end 214 within the bodily space (not shown), yet there is still an impediment to fluid leakage through tube 212 into hub 206. To allow the medical practitioner to reliably move needle cannula 204 from the first position to the third position without going past the third position into the second position, one or both of a first marker 260 and a second marker 262 are associated with shaft 220 in respective areas 264 and 266 thereof which are each sufficiently proximal of heel 230 so as to be within catheter 202, and advantageously within tube 212 as shown in FIG. 7, in the first position of needle cannula 204, but so that in the third position of needle cannula 204, first marker 260 is exposed beyond hub 206 and housing 234 such as just proximal of housing 234 and/or second marker 262 is exposed proximally of tube 212 and hub 206 but within housing 234 in juxtaposition to pass along edge(s) 248 of canting plate 240, all as seen in FIG. 9.

Tube 212 of catheter 202 extends a distance L1 from hub distal end 210 to tube distal end 214. Hub 206 and housing 234 have a combined length L2 between housing proximal end 238 and hub distal end 210, with hub 206 having a length L2H between proximal opening 208 and distal end 210 long enough for duckbill arms 242 to engage within hub 206 in the first position of needle cannula 204. Plate 240 is typically held within housing 234 such that through-hole 244 is spaced proximally of the distal end 210 of hub 206 by a distance L4. In accordance with the principles of the present invention, areas 264 and 266 are proximally spaced from heel 230 a respective distance L3V and L3T, which bear respective relationships to L1, L2, L2H and/or L4. To that end, L3V is advantageously greater than L2 and less than the sum of L1 plus L2, and more advantageously, less than L1 plus L2H. For many catheters, L3V may still more advantageously be less than L1, but for short catheters, may be greater than L1. Similarly, L3T is advantageously greater than L4 and less than the sum of L1 plus L4, and more advantageously less than L1. The foregoing advantageously places markers 260 and 262 within catheter 202, and possibly within catheter tube 212, in the first position of needle cannula 204, and such that they are exposed beyond at least catheter hub 206, with marker 262 further being exposed beyond housing 234, in the third position of needle cannula 204.

In the catheter insertion device 200 of in FIGS. 7-9, marker 260 is advantageously a visual marker and marker 262 is advantageously a tactile marker such that the medical practitioner may visualize emergence of marker 260 as it passes beyond (i.e., is exposed beyond) housing 234 and/or may feel a tactile sensation as marker 264 passes along edge(s) 248 of through-hole 244, whereat the medical practitioner should stop withdrawal of needle cannula 204 along the direction of arrow A to thus reliably position needle cannula 204 in the desired third position. At that position, the medical practitioner may then finalize placement of catheter tube 212, and particularly distal portion 254 and/or opening 214 thereof, within the bodily space, followed, advantageously, by withdrawal of needle cannula 204 to at least the second position shown in FIG. 8.

The visual marker 260 may be associated with area 264 by applying a visually perceptible material such as ink to the surface 250 of shaft 220 at or up to area 264 or by a visually perceptible surface affect at or up to area 264 by grinding, filing, etching, knurling, or otherwise altering the visually perceptible appearance of surface 250. Advantageously, marker 260 is not such as would create a noticeable tactile sensation as it passes along any of edges 248, 252, and further advantageously does not create an outward projection large enough to prevent proximal withdrawal of needle cannula 204 through plate 240 to the second position. Tactile marker 262 may be a disruption of surface 250 in area 266 which results in either an outwardly directed (relative to surface 250) projection, of an inwardly directed (relative to surface 250) recess or the like (such as a notch or gap, by way of example), or a roughening of surface 250 sufficient to interact with edge(s) 248 (but advantageously not edge 252), to create a tactile feedback to the user (not shown) as marker 262 passes along edge(s) 248. Where the disruption is an outwardly directed projection (not shown), it may be associated with area 266 by crimping or coining surface 250 thereat, by way of example, although the extent of outward projection is advantageously not so large as to prevent proximal withdrawal of needle cannula 204 through plate 240 into the second position. Where marker 262 is a roughening of surface 250 or is an inwardly directed recess or the like, it may be associated with area 266 by grinding, filing, etching, machining, or milling, by way of example. Also, while tactile marker 262 is described herein as providing a tactile response in passing along edge(s) 248, marker 262 could instead be adapted to cooperate with edge 252 to produce the tactile response, meaning that associated area 266 would be more distal than that shown and L4 would be measured relative to edge 252 rather than through-hole 244.

Use of safety catheter insertion device 200 is similar to both catheter insertion devices 10 and 70, except that withdrawal of the needle cannula 204 may be stopped at the third position by either seeing some portion of visual marker 260 exposed beyond housing 234 and/or feeling a tactile sensation by tactile marker 262 exposed beyond hub 206 and within housing 234 to pass along edge(s) 248.

By virtue of the foregoing, there is thus provided a catheter insertion device which provides the medical practitioner with the ability to reliably withdraw the needle cannula sufficiently to free the distal portion of the tube to flex so as to facilitate final placement of the tube in the bodily space while maintaining at least a portion of the shaft proximal of the sharp tip within a proximal portion of the tube to continue to impede fluid leakage.

While the present invention has been illustrated by the description of embodiments thereof, and while the embodiments have been described in considerable detail, it is not intended to restrict or in any way limit the scope of the appended claims to such detail. Additional advantages and modifications will readily appear to those skilled in the art. For example, while separate visual and tactile markers are shown, there could be one marker to serve both as a visual marker and as a tactile marker. In many catheter insertion devices, the needle cannula is hollow and thus has a fluid lumen therethrough. The marker(s) of the present invention advantageously will not extend through the needle shaft surface so as to open a fluid path into the fluid lumen thereat. Otherwise, as the marker becomes exposed beyond the tube, an alternate leakage path would be created. Further, while the markers are shown as being wholly beyond the catheter, catheter tube, catheter hub or housing as the context admits, it will be appreciated that as used herein, a marker is “exposed beyond” even if only a portion thereof is beyond, while the rest is not, and that “exposed beyond” is a positional relationship not necessarily requiring that the marker be visible to the user. The invention in its broader aspects is, therefore, not limited to the specific details, representative apparatus and method, and illustrative examples shown and described. Accordingly, departures may be made from such details without departing from the spirit or scope of the general inventive concept.

Having described the invention, what is claimed is:

1. A catheter insertion device comprising:
a catheter having a catheter hub and a catheter tube extending therefrom to a distal end;
a needle cannula having a needle shaft terminating in a sharp tip, the needle cannula having a first position with the shaft extending through the catheter hub and catheter tube such that the sharp tip is projecting out from the catheter tube, the needle cannula having a second position wherein the needle shaft is removed from the catheter tube, the needle cannula having a third position intermediate the first and second positions wherein the needle shaft extends through the catheter hub and into a proximal portion of the catheter tube such that the sharp tip is within the catheter tube and the shaft is not in a distal portion of the catheter tube; and
a marker associated with an area of the needle shaft such that the marker being within the catheter tube in the first position of the needle cannula and being exposed beyond the catheter tube in the third position of the needle cannula.

2. The catheter insertion device of claim 1 further comprising a safety device having a portion being resiliently urged against the shaft in at least the first and third positions of the needle cannula.

3. The catheter insertion device of claim 2, the marker being exposed beyond the safety device in the third position of the needle cannula.

4. The catheter insertion device of claim 3, the resiliently-urged portion of the safety device being within the catheter hub in the first and third positions of the needle cannula.

5. The catheter insertion device of claim 3, the resiliently-urged portion of the safety device being proximal of the catheter hub.

6. The catheter insertion device of claim 2, the resiliently-urged portion being urged against the shaft in the second position of the needle cannula.

7. The catheter insertion device of claim 2, the marker being a tactile marker and the marker passing along the resiliently-urged portion in the third position of the needle cannula, whereby to provide tactile feedback that the needle cannula is in the third position.

8. The catheter insertion device of claim 1, the marker being within the catheter hub in the third position of the needle cannula.

9. The catheter insertion device of claim 1, the marker being exposed beyond the catheter hub in the third position of the needle cannula.

10. The catheter insertion device of claim 9, the marker being a visual marker whereby to provide a visual indication that the needle cannula is in the third position.

11. The catheter insertion device of claim 9 further comprising a housing extending from the catheter hub.

12. The catheter insertion device of claim 11, the marker being exposed beyond the housing in the third portion of the needle cannula.

13. The catheter insertion device of claim 12, the marker being a visual marker whereby to provide a visual indication that the needle cannula is in the third position.

14. The catheter insertion device of claim 11 further comprising a safety device having a portion within the housing and being resiliently urged against the shaft in at least the first and third positions of the needle cannula, the marker being a tactile marker and the marker passing along the resiliently-urged portion in the third position of the needle cannula, whereby to provide tactile feedback that the needle cannula is in the third position.

15. The catheter insertion device of claim 14, the resiliently urged portion being urged against the shaft in the second position of the needle cannula.

16. The catheter insertion device of claim 1 further comprising a second marker associated with a second area of the needle shaft such that the second marker being within the catheter tube in the first position of the needle cannula and being exposed beyond the catheter tube in the third position of the needle cannula.

17. The catheter insertion device of claim 16, one of the markers further being exposed beyond the catheter hub in the third position of the needle cannula.

18. The catheter insertion device of claim 17, both of the markers being exposed beyond the catheter hub in the third position of the needle cannula.

19. The catheter insertion device of claim 16 further comprising a safety device having a portion being resiliently urged against the shaft in at least the first and third positions of the needle cannula.

20. The catheter insertion device of claim 19, one of the markers being a visual marker and being exposed beyond the safety device whereby to provide a visual indicator that the needle cannula is in the third position.

21. The catheter insertion device of claim 19, one of the markers being a tactile marker and passing along the resiliently-urged portion in the third position of the needle cannula, whereby to provide tactile feedback that the needle cannula is in the third position.

22. The catheter insertion device of claim 1, the marker being a visually perceptible material applied to the shaft.

23. The catheter insertion device of claim 1, the marker being a visually perceptible surface affect of the shaft.

24. The catheter insertion device of claim 1, the marker being a disruption of a surface of the shaft.

25. The catheter insertion device of claim 1, the marker being an outward projection

26. The catheter insertion device of claim 1, the marker being an inward projection.

27. The catheter insertion device of claim 1, the marker being a roughening of the surface.

28. The catheter insertion device of claim 1 further comprising a second marker associated with a second area of the needle shaft such that the second marker being within the catheter in the first position of the needle cannula and being exposed beyond the catheter hub in the third position of the needle cannula.

29. A catheter insertion device comprising:
a catheter having a catheter hub and a catheter tube extending therefrom to a distal end;
a needle cannula having a needle shaft terminating in a sharp tip, the needle cannula having a first position with the shaft extending through the catheter hub and catheter tube such that the sharp tip is projecting out from the catheter tube, the needle cannula having a second position wherein the needle shaft is removed from the catheter tube, the needle cannula having a third position intermediate the first and second positions wherein the needle shaft extends through the catheter hub and into a proximal portion of the catheter tube such that the sharp tip is within the catheter tube and the shaft is not in a distal portion of the catheter tube; and
a marker associated with an area of the needle shaft such that the marker being within the catheter in the first position of the needle cannula and being exposed beyond the catheter hub in the third position of the needle cannula.

30. A catheter insertion device comprising:
a catheter having a catheter hub of length L2 between a distal end and a proximal end, and a catheter tube extending distally from the catheter hub distal end a distance L1 to a tube distal end;
a needle cannula having a needle shaft terminating in a sharp tip extending distally from a heel, the needle cannula being moveable within the catheter; and
a marker associated with an area of the needle shaft, the area being proximally spaced from the heel a distance L3, L3 being greater than L2 and less than L1 plus L2.

31. The catheter insertion device of claim 30, L3 being greater than L1.

32. The catheter insertion device of claim 30, L3 being less than L1.

33. A catheter insertion device comprising:
a catheter having a catheter hub of length L2 between a distal end and a proximal end, and a catheter tube extending distally from the catheter hub distal end a distance L1 to a tube distal end:
a safety device having a resiliently urged portion situated relative to the catheter hub a distance L4 proximally of the catheter hub distal end;
a needle cannula having a needle shaft terminating in a sharp tip extending distally from a heel, the needle cannula being moveable within the catheter and relative to the resiliently urged portion; and
a marker associated with an area of the needle shaft, the area being proximally spaced from the heel a distance L3T, L3T being greater than L4 and less than L1 plus L4.

34. The catheter insertion device of claim 33, L3T being less than L1.

35. The catheter insertion device of claim 33 further comprising a second marker associated with a second area of the needle shaft, the second area being proximally spaced from the heel a distance L3V, L3V being greater than L2 and less than L1 plus L2.

36. The catheter insertion device of claim 35, L3V being greater than L1.

37. The catheter insertion device of claim 35, L3V being less than L1.

38. The catheter insertion device of claim 35, L3V being greater than L3T.

39. The catheter insertion device of claim 33, the resiliently urged portion being situated within the catheter hub.

40. A catheter insertion device comprising:
a catheter having a catheter hub of length L2H between a distal end and a proximal end, and a catheter tube extending distally from the catheter hub distal end a distance L1 to a tube distal end:
a housing selectively coupled to and extending proximally of the catheter hub to a proximal end, the catheter hub and housing, when coupled, having a length L2 between the housing proximal end and the catheter hub distal end;
a safety device having a resiliently urged portion situated within the housing relative to the catheter hub a distance L4 proximally of the catheter hub distal end when the catheter hub and housing are coupled;
a needle cannula having a needle shaft terminating in a sharp tip extending distally from a heel, the needle cannula being moveable within the catheter and housing and relative to the resiliently urged portion; and
a marker associated with an area of the needle shaft, the area being proximally spaced from the heel a distance L3T, L3T being greater than L4 and less than L1 plus L4.

41. The catheter insertion device of claim 40, L3T being less than L1.

42. The catheter insertion device of claim 40 further comprising a second marker associated with a second area of the needle shaft, the second area being proximally spaced from the heel a distance L3V, L3V being greater than L2 and less than L1 plus L2.

43. The catheter insertion device of claim 42, L3V being less than L1 plus L2H.

44. The catheter insertion device of claim 42, L3V being greater than L1.

45. The catheter insertion device of claim 42, L3V being less than L1.

46. The catheter insertion device of claim 42, L3V being greater than L3T.

47. A method of making a catheter insertion device including a catheter having a catheter hub and a catheter tube extending therefrom and a needle cannula having a shaft terminating in a sharp tip end comprising:
associating a marker with an area of the shaft which would place the marker within the catheter tube with the sharp tip exposed beyond the catheter tube and which would place the marker exposed beyond the catheter tube with the needle shaft and sharp tip within a proximal portion of the catheter tube and not in a distal portion of the catheter tube; and
inserting the needle cannula through the catheter hub and the catheter tube until a sharp tip of the needle cannula is exposed beyond the catheter tube such that the marker is within the catheter tube.

48. The method of claim 47 wherein the area would place the marker exposed beyond the catheter hub with the needle shaft and sharp tip within a proximal portion of the catheter tube and not in a distal portion of the catheter tube.

49. The method of claim 47 wherein the area would place the marker within the catheter hub with the needle shaft and sharp tip within a proximal portion of the catheter tube and not in a distal portion of the catheter tube.

50. The method of claim 47 wherein the catheter insertion device further includes a safety device having a resiliently urged portion, the method further comprising inserting the needle cannula such that the shaft passes along the resiliently-urged portion.

51. The method of claim 50 wherein the area would pass the marker along the resiliently-urged portion with the needle shaft and sharp tip within a proximal portion of the catheter tube and not in a distal portion of the catheter tube.

52. The method of claim 50 further comprising applying a second marker to a second area of the shaft which would place the second marker within the catheter tube with the sharp tip exposed beyond the catheter tube and which would place the second marker exposed beyond the safety device with the needle shaft and sharp tip within a proximal portion of the catheter tube and not in a distal portion of the catheter tube.

53. The method of claim 50 further comprising applying a second marker to a second area of the shaft which would place the second marker within the catheter with the sharp tip exposed beyond the catheter tube and which would place the second marker exposed beyond the safety device with the needle shaft and sharp tip within a proximal portion of the catheter tube and not in a distal portion of the catheter tube.

54. The method of claim 47 further comprising applying a second marker to a second area of the shaft which would place the marker within the catheter with the sharp tip exposed beyond the catheter tube and which would place the marker exposed beyond the catheter hub with the needle shaft and sharp tip within a proximal portion of the catheter tube and not in a distal portion of the catheter tube.

55. A method of making a catheter insertion device including a catheter having a catheter hub and a catheter tube extending therefrom and a needle cannula having a shaft terminating in a sharp tip end comprising:
associating a marker with an area of the shaft which would place the marker within the catheter with the sharp tip exposed beyond the catheter tube and which would place the marker exposed beyond the catheter hub with the needle shaft and sharp tip within a proximal portion of the catheter tube and not in a distal portion of the catheter tube; and
inserting the needle cannula through the catheter hub and the catheter tube until a sharp tip of the needle cannula is exposed beyond the catheter tube such that the marker is within the catheter.
